(19) United States
(12) Patent Application Publication
(10) Pub. No.: US 2024/0084100 A1
(43) Pub. Date: Mar. 14, 2024

(54) PLASTICIZER COMPOSITION AND VINYL CHLORIDE RESIN COMPOSITION COMPRISING THE SAME

(71) Applicant: HANWHA SOLUTIONS CORPORATION, Seoul (KR)

(72) Inventors: Hee-La KWAK, Daejeon (KR); Jaesong KIM, Daejeon (KR); Myung-Ik YOO, Daejeon (KR)

(21) Appl. No.: 18/259,282

(22) PCT Filed: Jan. 4, 2022
(86) PCT No.: PCT/KR2022/000099

§ 371 Date: Jun. 26, 2023

(30) Foreign Application Priority Data
Feb. 5, 2021 (KR) 10-2021-0016868

(51) Int. Cl. C08K 5/12; C08K 3/22; C08K 5/11

(52) U.S. Cl. CPC C08K 5/12; C08K 3/22; C08K 5/11; C08K 2003/2241; C08K 2201/014

(57) ABSTRACT

The present disclosure relates to a plasticizer composition having excellent plasticization efficiency and migration resistance with significantly improved weather resistance and cold resistance, and a vinyl chloride resin composition including the same.

### CROSS-REFERENCE TO RELATED APPLICATION(S)

This application claims the benefits of Korean Patent Application No. 10-2021-0016868 filed on Feb. 5, 2021 with the Korean Intellectual Property Office, the disclosure of which is incorporated herein by reference in its entirety.

The present disclosure relates to a plasticizer composition having excellent plasticization efficiency and migration resistance with significantly improved weather resistance and cold resistance, and a vinyl chloride resin composition including the same.

### BACKGROUND OF ART

### Technical Field

A vinyl chloride-based resin includes a homopolymer of vinyl chloride and a copolymer containing 50% or more of vinyl chloride, and is one of five general-purpose thermoplastic resins prepared by suspension polymerization and emulsion polymerization.

The vinyl chloride-based resin is used in combination with auxiliary materials such as plasticizers, stabilizers, fillers, and foaming agents in order to impart functionality. The plasticizer is added to improve processability by imparting flexibility to vinyl chloride-based resins, and a phthalate-based plasticizer such as DEHP (Di-2-EthylHexyl Phthalate), DINP (Di-IsoNonyl Phthalate), DIDP (Di-Iso-Decyl Phthalate), BBP (Butyl Benzyl Phthalate), and DBP (Di-n-butyl phthalate) has been widely used.

Although the phthalate-based plasticizer has excellent plasticization efficiency, it is harmful to the human body, so its use has recently been regulated both at home and abroad. Accordingly, a terephthalate-based plasticizer, an epoxide-based plasticizer, a vegetable oil-based plasticizer, a cyclohexane-based plasticizer, and the like have been proposed as a material that can replace the phthalate-based plasticizer. A typical eco-friendly plasticizer is dioctyl terephthalate (DOTP), but the DOTP plasticizer has problems in migration, poor weather resistance against ultraviolet rays, and a rapid increase in viscosity in winter.

In particular, since the vinyl chloride resin composition for manufacturing PVC hoses used for agriculture, horticulture, and household purposes contains a large amount of plasticizers in order to secure flexibility, it is further required to improve physical properties of the plasticizer. Therefore, it is required to develop an eco-friendly plasticizer composition that is not easily aged by ultraviolet rays, heat, and low temperature, has excellent migration resistance, and exhibits high plasticization efficiency.

### DETAILED DESCRIPTION OF THE INVENTION

### Technical Problem

In order to solve the above problems, there is provided an eco-friendly plasticizer composition having excellent plasticization efficiency and migration resistance with significantly improved weather resistance and cold resistance.

In addition, there is provided a vinyl chloride resin composition including the plasticizer composition.

In addition, there is provided a molded product including the vinyl chloride resin composition.

### Technical Solution

In order to solve the above problems, there is provided a plasticizer composition including di(2-ethylhexyl)cyclohexane-1,4-dicarboxylate, dioctyl adipate, and a compound represented by the following Chemical Formula 1:

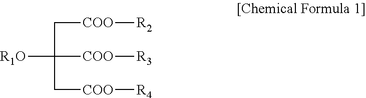

in Chemical Formula 1,
R₁ is hydrogen or acetyl, and
R₂ to R₄ are each independently C2-8 alkyl.

In addition, there is provided a vinyl chloride resin composition including the above-described plasticizer composition and a molded product including the above-described vinyl chloride resin composition.

### Advantageous Effects

The plasticizer composition according to the present disclosure is eco-friendly because it does not contain a phthalate-based compound, and exhibits excellent plasticization efficiency and plasticizer migration resistance with significantly improved weather resistance and cold resistance.

Therefore, the vinyl chloride resin composition including the plasticizer composition of the present disclosure exhibits excellent plasticization performance with a smaller amount of use, and maintains good physical properties without plasticizer migration even in ultraviolet rays, high temperature and low temperature environments. Therefore, it can be suitably used in fields requiring high flexibility, weather resistance and high cold resistance, and in particular, can be suitably used for manufacturing soft hoses mainly used outdoors.

### DETAILED DESCRIPTION OF THE EMBODIMENTS

The terminology used herein is for the purpose of describing particular embodiments only and is not intended to be limiting of the invention. The singular forms are intended to include the plural forms as well, unless the context clearly indicates otherwise.

As the present invention can be variously modified and have various forms, specific embodiments thereof are shown by way of examples and will be described in detail. However, it is not intended to limit the present invention to the particular form disclosed and it should be understood that the present invention includes all modifications, equivalents, and replacements within the idea and technical scope of the present invention.

Hereinafter, the plasticizer composition and the vinyl chloride resin composition including the same according to specific embodiments of the present invention will be described in more detail.

Plasticizer Composition

The plasticizer composition according to the present disclosure is invented based on the fact that plasticization efficiency and migration resistance of the plasticizer are excellent due to the interaction of three specific plasticizers while weather resistance and cold resistance can be significantly improved.

The plasticizer composition according to one embodiment of the present disclosure includes di(2-ethylhexyl)cyclohexane-1,4-dicarboxylate, dioctyl adipate, and the compound represented by the Chemical Formula 1 above.

The di(2-ethylhexyl)cyclohexane-1,4-dicarboxylate is a compound represented by the following Chemical Formula 2, and is also referred to as DEHCH in English abbreviation:

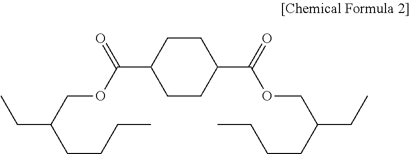 [SMILES: CCCCC(CC)COC(=O)C1CCC(C(=O)OCC(CC)CCCC)CC1]

DEHCH has low viscosity at room temperature and low temperature, so it can achieve excellent coating properties, has a fast gelling rate, and has excellent plasticization efficiency. In particular, generation of volatile organic compounds can be minimized and biotoxicity is low compared to existing phthalate-based plasticizers.

However, there are limitations when using DEHCH alone in fields requiring high cold resistance. In the present disclosure, dioctyl adipate and the compound of Chemical Formula 1 are used together with DEHCH.

The dioctyl adipate is a compound represented by the following Chemical Formula 3 below, and is also referred to as DOA in English abbreviation:

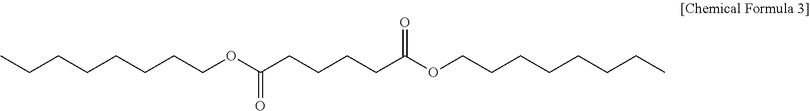 [SMILES: CCCCCCCCOC(=O)CCCCC(=O)OCCCCCCCC]

DOA has excellent thermal stability and cold resistance. However, when DOA is used alone, there is a problem in that migration resistance and processability are relatively poor, and the unit price is high, which increases production cost. Accordingly, the above problem is solved by using DEHCH and the compound of Chemical Formula 1 together in the present disclosure.

Meanwhile, since the plasticizer composition containing DEHCH, DOA, and the compound of Chemical Formula 1 has excellent plasticizer migration resistance, it can be used in a resin composition to increase the lifespan of a product.

The compound represented by Chemical Formula 1 is an ester compound of citric acid and C2-8 alcohol, and prevents the plasticizer from leaking out of the vinyl chloride-based resin due to interactions such as hydrogen bonding between the ester groups, which is not theoretically limited.

In Chemical Formula 1, it is preferable that R₂ to R₄ are each independently butyl or octyl.

Representative examples of the compound represented by Chemical Formula 1 are tributylcitrate, tri(butyloctyl)citrate, trioctylcitrate, and acetyltributylcitrate.

Preferably, the compound represented by Chemical Formula 1 is tributyl citrate (TBC). TBC has excellent processability due to its small molecular weight, and is particularly excellent in improving migration resistance.

In a preferred embodiment of the present disclosure, DEHCH may be included in an amount of 40 parts by weight or more, preferably 50 parts by weight or more, or 60 parts by weight or more based on 100 parts by weight of the plasticizer composition. In addition, DEHCH may be included in an amount of 90 parts by weight or less, 80 parts by weight or less, or 70 parts by weight or less based on 100 parts by weight of the plasticizer composition. When the content of DEHCH satisfies the above range, excellent plasticization efficiency (low hardness) can be achieved, initial colorability is excellent to easily implement color, and high processability can be exhibited.

In addition, DOA may be included in an amount of 5 parts by weight or more, 10 parts by weight or more, or 15 parts by weight or more, and 50 parts by weight or less, 30 parts by weight or less, or 20 parts by weight or less based on 100 parts by weight of the plasticizer composition. When the content of DOA is less than 5 parts by weight based on 100 parts by weight of the plasticizer composition, cold resistance and heat stability of the plasticizer composition may be deteriorated, and when it exceeds 50 parts by weight, processability may be deteriorated.

In addition, the compound represented by Chemical Formula 1 may be included in an amount of 5 parts by weight or more, 10 parts by weight or more, or 15 parts by weight or more, and 50 parts by weight or less, 30 parts by weight or less, or 20 parts by weight or less based on 100 parts by weight of the plasticizer composition. When the content of the compound represented by Chemical Formula 1 is less than 5 parts by weight based on 100 parts by weight of the plasticizer composition, the plasticizer migration resistance may not be sufficiently improved, and when it exceeds 50 parts by weight, the effect of improving weather resistance, cold resistance, and thermal stability may decrease.

Accordingly, the plasticizer composition according to a preferred embodiment of the present disclosure may include 40 to 90 parts by weight of di(2-ethylhexyl)cyclohexane-1,4-dicarboxylate, 5 to 50 parts by weight of dioctyl adipate, and 5 to 50 parts by weight of the compound represented by Chemical Formula 1. Herein, the compound represented by Chemical Formula 1 may be tributyl citrate.

The plasticizer composition according to another preferred embodiment may include 60 to 90 parts by weight of di(2-ethylhexyl)cyclohexane-1,4-dicarboxylate, 5 to 20 parts by weight of dioctyl adipate, and 5 to 20 parts by weight of the compound represented by Chemical Formula 1. Herein, the compound represented by Chemical Formula 1 may be tributyl citrate.

Meanwhile, the plasticizer composition according to one embodiment of the present disclosure may not include other plasticizers other than the aforementioned DEHCH, DOA, and the compound represented by Chemical Formula 1. That is, the plasticizer composition may consist of only DEHCH, DOA, and the compound represented by Chemical Formula 1.

Herein, the compound represented by Chemical Formula 1 may be tributyl citrate, and in this case, the plasticizer composition may consist of only DEHCH, DOA, and TBC.

The plasticizer composition of the present disclosure maintains excellent plasticization performance under light, heat, and low temperature conditions and has high weather resistance and cold resistance such as little change in color and physical properties compared to the case of using anyone alone or using only two of DEHCH, DOA, and the compound represented by Chemical Formula 1 in combination due to the synergistic effect of DEHCH, DOA, and the compound represented by Chemical Formula 1. In addition, the plasticizer composition has high plasticization efficiency and excellent plasticizer migration resistance even under harsh conditions while being eco-friendly because it does not contain a phthalate-based compound.

Vinyl Chloride Resin Composition

In addition, according to another embodiment of the present disclosure, there is provided a vinyl chloride resin composition including a vinyl chloride-based resin and the above-described plasticizer composition.

More specifically, the vinyl chloride resin composition may include 50 to 90 parts by weight, 60 to 90 parts by weight, or 70 to 80 parts by weight of the plasticizer composition based on 100 parts by weight of the vinyl chloride-based resin.

The vinyl chloride resin composition not only exhibits excellent weather resistance and cold resistance, but also further improves properties such as color, plasticization efficiency, and plasticizer migration by including the plasticizer composition according to the present disclosure.

Throughout the present disclosure, the vinyl chloride-based resin refers to a polymer obtained by polymerizing a vinyl chloride-based monomer alone or a copolymer obtained by copolymerizing a vinyl chloride-based monomer and a comonomer copolymerizable therewith. In addition, conventional polymerization methods known in the art to which the present invention pertains such as suspension polymerization, bulk polymerization, emulsion polymerization, and seed emulsion polymerization may be used after mixing with a suspending agent, a buffer, a polymerization initiator, and the like.

Other monomers copolymerizable with the aforementioned vinyl chloride monomer may include, for example, vinyl ester-based monomer including ethylene vinyl acetate monomer and vinyl propionate monomer; olefinic monomer including ethylene, propylene, isobutyl vinyl ether, and halogenated olefin; methacrylic acid ester-based monomer including methacrylic acid alkyl ester; maleic anhydride monomer; acrylonitrile monomer; styrene monomer; halogenated polyvinylidene, etc., and a copolymer with the vinyl chloride monomer may be prepared by mixing one or more thereof. However, the present disclosure is not limited to the above-mentioned monomers, and monomers generally used to form a copolymer through a polymerization reaction with a vinyl chloride monomer in the art to which the present invention pertains can be used without particular limitation according to the physical properties or uses of the vinyl chloride resin composition required during production.

Preferably, the vinyl chloride-based resin may be polyvinyl chloride (PVC) with a polymerization degree of 500 to 3,000, but is not limited thereto.

The vinyl chloride resin composition may further include at least one additive selected from the group consisting a stabilizer, a filler, a flame retardant, a lubricant, and a pigment. The additive may be appropriately selected according to physical properties to be improved in the vinyl chloride resin composition.

The stabilizer is added for the purpose of preventing changes in the various physical properties caused by separation of HCl from the vinyl chloride-based resin to form a polyene structure, which is a chromophore, causing cutting and cross-linking of the main chain, and includes at least one selected from the group consisting of Cd—Zn-based compounds, Ca—Zn-based compounds, K—Zn-based compounds, Ba—Zn-based compounds, organic Tin-based compounds; metallic soap-based compounds, phenol-based compounds, phosphoric acid ester-based compounds and phosphorous acid ester-based compounds.

More specific examples of the stabilizer which may be used in the present disclosure may include Cd—Zn-based compounds; Ca—Zn-based compounds; K—Zn-based compounds; Ba—Zn-based compounds; organic Tin-based compounds such as mercaptide-based compounds, maleic acid-based compounds or carboxylic acid-based compounds; metallic soap-based compounds such as Mg-stearate, Ca-stearate, Pb-stearate, Cd-stearate, or Ba-stearate; phenol-based compounds; phosphoric acid ester-based compounds; phosphorous acid ester-based compounds, etc. It is optionally included according to the purpose of use.

The stabilizer is preferably included in an amount of 0.5 to 7 parts by weight, more preferably 1 to 4 parts by weight based on 100 parts by weight of the vinyl chloride-based resin. When the content of the stabilizer is less than 0.5 parts by weight, there is a problem of poor thermal stability, and when it exceeds 7 parts by weight, there is a problem of poor processability.

The filler is used for the purpose of improving productivity, and dry touch feeling of the vinyl chloride resin composition, and includes at least one selected from the group consisting of calcium carbonate, talc, titanium dioxide, kaolin, silica, alumina, magnesium hydroxide, and clay.

In the vinyl chloride resin composition according to the present disclosure, the filler may be included in an amount of 10 to 150 parts by weight, more preferably 50 to 130 parts by weight based on 100 parts by weight of the vinyl chloride-based resin. When the filler is included in an amount of less than 10 parts by weight, there is a problem in that dimensional stability and economic feasibility are lowered, and when it is included in an amount of more than 130 parts by weight, there is a problem in that processability is lowered.

Examples of the flame retardant may include antimony trioxide; brominated flame retardant such as tetrabromo dioctylphthalate; chlorinated paraffin; zinc borate; phosphoric acid ester; molybdenum compounds such as zinc molybdate and ammonium octamolybdate; tin compounds such as zinc hydroxystannate or tartaric acid; etc. The flame retardant may be included in an amount of 1 part by weight to 200 parts by weight, or 3 parts by weight to 150 parts by weight based on 100 parts by weight of the vinyl chloride resin.

The pigment may be carbon black, titanium dioxide (TiO₂), iron oxide-based, cadmium-based, chromium-based, zinc-based, or titanium-based pigments. Examples of the pigment may include zinc oxide; chromium red; chrome green; phthalocyanine green; chrome yellow; FeO—OH; ultra marine blue; milori blue; phthalocyanine blue; Fe₂O₃; molybdenum red; and metal powder pigments. However, it is not limited thereto, and organic pigments, inorganic pigments, or any additives used for the purpose of expressing color or improving concealment are considered to be used as the pigments.

The pigment may be included in an amount of 0.1 to 10 parts by weight or 1 to 15 parts by weight based on 100 parts by weight of the vinyl chloride resin. When the pigment is included in an amount of less than 0.1 parts by weight, color implementation is difficult, and when the amount exceeds 10 parts by weight, mechanical properties may be deteriorated.

The vinyl chloride resin composition according to the present disclosure may be prepared by a method generally known in the art using a vinyl chloride-based resin, the plasticizer composition, and optionally an additive, and the method is not particularly limited.

The vinyl chloride resin composition has excellent weather resistance and cold resistance, excellent color, and high plasticization efficiency. In addition, since plasticizer migration is improved and surface damage by the plasticizer migration is reduced even when exposed to sunlight, heat, etc., the composition can be suitably used for products mainly used outdoors. Specifically, the vinyl chloride resin composition may be used for manufacturing soft hoses for agriculture, horticulture, and household submersible pumps.

Meanwhile, according to another aspect of the present disclosure, there is provided a molded product including the above-described vinyl chloride resin composition. The molded product may be manufactured by additionally adding an additive such as a stabilizer, a filler, and/or a flame retardant to the vinyl chloride resin composition according to its use.

The molded product may be a soft hose for agriculture, horticulture, household submersible pumps, and the like.

Hereinafter, the present invention will be described in more detail with the following examples. However, the following examples are only for illustrating the present invention, and the scope of the present invention is not limited thereto.

### EXAMPLES

Preparation of Plasticizer Composition and Vinyl Chloride Resin Composition

### Example 1

A plasticizer composition was prepared by mixing 70 parts by weight of di(2-ethylhexyl)cyclohexane-1,4-dicarboxylate (DEHCH), 15 parts by weight of dioctyl adipate (DOA), and 15 parts by weight of tributyl citrate (TBC).

100 parts by weight of polyvinyl chloride (Hanwha Solution P-1000F, polymerization degree 1000±50, bulk density 0.55±0.04 g/cm³), 80 parts by weight of the plasticizer composition, 20 parts by weight of a filler (Omya 1t), 1 part by weight of Cd/Zn-based stabilizer were mixed to prepare a vinyl chloride resin composition.

### Example 2

A plasticizer composition was prepared by mixing 60 parts by weight of di(2-ethylhexyl)cyclohexane-1,4-dicarboxylate (DEHCH), 20 parts by weight of dioctyl adipate (DOA), and 20 parts by weight of tributyl citrate (TBC).

Then, a vinyl chloride resin composition was prepared in the same manner as in Example 1.

### Comparative Example 1

A plasticizer composition was prepared by mixing 80 parts by weight of di(2-ethylhexyl)cyclohexane-1,4-dicarboxylate (DEHCH), and 20 parts by weight of dioctyl adipate (DOA).

Then, a vinyl chloride resin composition was prepared in the same manner as in Example 1.

### Comparative Example 2

A plasticizer composition was prepared by mixing 60 parts by weight of di(2-ethylhexyl)cyclohexane-1,4-dicarboxylate (DEHCH), and 20 parts by weight of tributyl citrate (TBC).

Then, a vinyl chloride resin composition was prepared in the same manner as in Example 1.

### Comparative Example 3

A vinyl chloride resin composition was prepared in the same manner as in Example 1 using dioctyl terephthalate (di(2-ethylhexyl)terephthalate, DOTP) as a plasticizer.

### Comparative Example 4

A vinyl chloride resin composition was prepared in the same manner as in Example 1 using diisononyl phthalate (DINP) as a plasticizer.

### Comparative Example 5

A vinyl chloride resin composition was prepared in the same manner as in Example 1 using di(2-ethylhexyl)cyclohexane-1,4-dicarboxylate (DEHCH) as a plasticizer.

### Comparative Example 6

A vinyl chloride resin composition was prepared in the same manner as in Example 1 using tributyl citrate (TBC) as a plasticizer.

### Experimental Examples

(1) Hardness

The vinyl chloride resin composition was sequentially subjected to roll mill processing (170° C., 3 minutes) and press processing (180° C., 8 minutes) to prepare a flat sheet having a thickness of 6 mm.

In accordance with the ASTM D2240 method, the needle of the hardness tester (Shore D Type) was completely lowered into one part of the specimen and the hardness value was read after 5 seconds. After testing three parts for each specimen, the average value thereof was taken and used as an indicator of the plasticization efficiency.

(2) Plasticizer Migration

The plasticizer migration was evaluated as follows.

The prepared vinyl chloride resin composition was sequentially subjected to roll mill processing (170° C., 3 minutes) and press processing (180° C., 8 minutes) to prepare a flat sheet having a thickness of 2 mm. The prepared flat sheet was cut into a circular specimen having a diameter of about 4 cm. An oil paper for oil absorption made of polypropylene was placed on the top and bottom of the specimen, and left for 7 days at 60° C. under a load of 5 kg to promote plasticizer migration. After the completion of the plasticizer migration test, a weight change was measured for the specimen and the oil paper.

The weight change of the specimen was calculated as [(weight change of specimen/weight of specimen before test)*100], and the weight change of the oil paper was calculated as [(weight change of oil paper/weight of oil paper before test)*100]. The weight decrease of the specimen is the same as the weight increase of the oil paper, so the plasticizer migration was evaluated only by the weight change of the specimen in this test.

(3) Thermal Stability

A sheet obtained by roll milling the vinyl chloride resin composition (170° C., 3 minutes) was cut into a strap shape of 15 mm×480 mm and placed in a 190° C. Mathis oven. The specimen placed in the oven was set to be discharged out of the oven at 15 mm/5 minutes. The heat resistance time (minutes) was calculated with the length from the end of the test to the start of discoloration (length that is not carbonized, mm).

(4) Yellowness (YI) and Weather Resistance (ΔYI)

The vinyl chloride resin composition was sequentially subjected to roll mill processing (170° C., 3 minutes) and press processing (180° C., 8 minutes) to prepare a flat sheet having a thickness of 6 mm.

For the specimen prepared above, a yellow index (YI, yellowness) was measured according to ASTM E313. Then, the specimen was put into an accelerated weathering tester (ASTM G154, UVB 313), and exposed for 300 hours, followed by confirming whether cracks were generated on the surface and measuring YI.

(5) Cold Resistance (Low Brittleness Temp, LTB)

The prepared vinyl chloride resin composition was sequentially subjected to roll mill processing (170° C., 3 minutes) and press processing (180° C., 8 minutes) to prepare a flat sheet having a thickness of 2 mm, and a specimen was prepared according to ASTM D746 using the flat sheet. Cold resistance was evaluated by measuring the temperature at which 50% of the specimen to be measured was damaged when an impact was applied at a low temperature using a brittleness temperature tester.

Specifically, when the temperature at which 50% of the specimen to be measured was damaged was −60° C. or less, it was evaluated as excellent, when it was more than −60° C. to −50° C. or less, it was evaluated as very good, when it was more than −50° C. to −40° C. or less, it was evaluated as good, and when it was more than −40° C., it was evaluated as poor.

(6) Tensile Strength

A specimen was drawn in a cross head speed of 200 mm/min (1 T) using a test apparatus of U.T.M. (manufacturer: Instron, model name: 4466) according to ASTM D638, and a point where the specimen is cut was measured. Then, the tensile strength was calculated as follows:

Tensile strength (kgf/mm²)=load value (kgf)/thickness (mm)×width (mm)

(7) Elongation Rate

A specimen was drawn in a cross head speed of 200 mm/min (1 T) using the U.T.M. according to ASTM D638, and a point where the specimen is cut was measured. Then, the elongation rate was calculated as follows:

Elongation rate (%)=[length of specimen after elongation/initial length of specimen]×100

TABLE 1

| | Example 1 | Example 2 |
|---|---|---|
| Plasticizer composition | DEHCH:DOA:TBC = 70:15:15 | DEHCH:DOA:TBC = 60:20:20 |
| Hardness | 56 | 56 |
| Plasticizer migration | 0.2 | 0.2 |
| (%) | | |
| Thermal stability (min) | 40 | 39 |
| YI | 12.0 | 12.9 |
| Weather resistance (ΔYI) | 45 | 46 |
| Cold resistance (LTB) | excellent | excellent |
| | (−60° C. or less) | (−60° C. or less) |
| Tensile strength (kg/cm²) | 110 | 111 |
| Elongation rate (%) | 420 | 410 |

TABLE 2

| | Comp. | Comp. | Comp. | Comp. | Comp. | Comp. |
|---|---|---|---|---|---|---|
| | Example 1 | Example 2 | Example 3 | Example 4 | Example 5 | Example 6 |
| Plasticizer | DEHCH:DOA = | DEHCH:TBC = | DOTP | DINP | DEHCH | TBC |
| composition | 80:20 | 80:20 | | | | |
| Hardness | 58 | 56 | 63 | 61 | 58 | 58 |
| Plasticizer | 0.3 | 0.1 | 0.6 | 0.1 | 0.3 | 0.1 |
| migration | | | | | | |
| (%) | | | | | | |
| Thermal | 42 | 36 | 45 | 36 | 38 | 34 |
| stability | | | | | | |
| (min) | | | | | | |
| YI | 10.5 | 12.5 | 15.2 | 13.5 | 9.8 | 13.9 |
| Weather | 52 | 60 | 62 (surface | 47 (surface | 45 | 70 |
| resistance | | | crack) | crack) | | |
| (ΔYI) | | | | | | |
| Cold | very good | good | good | poor | very good | good |
| resistance | (−58° C.) | (−48° C.) | (−45° C.) | (−35° C.) | (−55° C.) | (−40° C.) |
| (LTB) | | | | | | |
| Tensile | 105 | 109 | 115 | 118 | 103 | 110 |
| strength | | | | | | |
| (kg/cm²) | | | | | | |
| Elongation | 379 | 410 | 420 | 430 | 460 | 425 |
| rate (%) | | | | | | |

Referring to Tables 1 and 2, it was confirmed that the plasticizer compositions of Examples including DEHCH, DOA, and TBC had plasticizer migration, thermal stability, tensile strength and elongation rate equal to or higher than conventional DOTP or DINP plasticizers, while exhibiting higher plasticization efficiency, and in particular, remarkably improved weather resistance and cold resistance.

In addition, it was confirmed that the plasticizer compositions of Examples showed significantly improved cold resistance compared to the case where only some of the three plasticizers were used, while having excellent plasticizer migration resistance and thermal stability

From these results, it was confirmed that the plasticizer compositions of the present disclosure could be suitably used in fields requiring high cold resistance and weather resistance, such as outdoor soft hoses.

1. A plasticizer composition comprising di(2-ethylhexyl)cyclohexane-1,4-dicarboxylate, dioctyl adipate, and a compound represented by the following Chemical Formula 1: 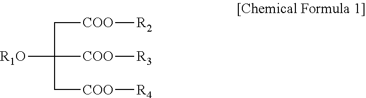
in Chemical Formula 1,
R₁ is hydrogen or acetyl, and
R₂ to R₄ are each independently C2-8 alkyl.

2. The plasticizer composition of claim 1,
wherein R₂ to R₄ are each independently butyl or octyl.

3. The plasticizer composition of claim 1,
wherein the compound represented by the Chemical Formula 1 is tributyl citrate, tri(butyloctyl)citrate, trioctyl citrate or acetyl tributyl citrate.

4. The plasticizer composition of claim 1,
wherein the plasticizer composition comprises 40 to 90 parts by weight of di(2-ethylhexyl)cyclohexane-1,4-dicarboxylate, 5 to 50 parts by weight of dioctyl adipate, and 5 to 50 parts by weight of the compound represented by the Chemical Formula 1.

5. The plasticizer composition of claim 1,
wherein the plasticizer composition comprises 60 to 90 parts by weight of di(2-ethylhexyl)cyclohexane-1,4-dicarboxylate, 5 to 20 parts by weight of dioctyl adipate, and 5 to 20 parts by weight of the compound represented by the Chemical Formula 1.

6. A vinyl chloride resin composition comprising a vinyl chloride-based resin, and the plasticizer composition according to claim 1.

7. The vinyl chloride resin composition of claim 6,
wherein the plasticizer composition is included in an amount of 50 to 90 parts by weight based on 100 parts by weight of the vinyl chloride-based resin.

8. The vinyl chloride resin composition of claim 6,
further comprising at least one additive selected from the group consisting of a stabilizer, a filler, and titanium dioxide.

9. The vinyl chloride resin composition of claim 8,
wherein the additive is included in an amount of 10 to 30 parts by weight based on 100 parts by weight of the vinyl chloride-based resin.

10. A molded product comprising the vinyl chloride resin composition of claim 6.

11. The molded product of claim 10,
wherein the molded product is a soft hose.
